(19) United States
(12) Patent Application Publication
(10) Pub. No.: US 2023/0181817 A1
(43) Pub. Date: Jun. 15, 2023

(54) INTRAVENOUS SET CORKSCREW HAND PUMP

(71) Applicant: CareFusion 303, Inc., San Diego, CA (US)

(72) Inventor: Kelly Kloster HON, Del Mar, CA (US)

(21) Appl. No.: 17/982,489

(22) Filed: Nov. 7, 2022

Related U.S. Application Data
(60) Provisional application No. 63/287,638, filed on Dec. 9, 2021.

(51) Int. Cl. A61M 5/14; A61M 5/142; A61M 5/168

(52) U.S. Cl. CPC A61M 5/1411; A61M 5/1424; A61M 5/16886; A61M 5/16877

(57) ABSTRACT

An IV set corkscrew hand pump includes a body, a corkscrew member disposed in the body, an inlet port disposed on a first end of the body, an outlet port disposed on a second end of the body and a drive mechanism coupled to the corkscrew member. The drive mechanism is configured to cause the corkscrew mechanism to turn within the body to increase fluid flow downstream in an IV set. IV sets with IV set corkscrew hand pumps and methods of operating IV set corkscrew hand pumps are also provided.

### CROSS-REFERENCE TO RELATED APPLICATIONS

This application claims the benefit of priority under 35 U.S.C. § 119 to U.S. Provisional Patent Application Ser. No. 63/287,638, entitled “INTRAVENOUS SET CORKSCREW HAND PUMP,” filed on Dec. 9, 2021, the entirety of which is incorporated herein by reference.

### TECHNICAL FIELD

The present disclosure generally relates to intravenous (IV) sets, in particular to hand operated and mechanical corkscrew hand pumps for IV sets.

### BACKGROUND

Medical treatments often include the infusion of a medical fluid (e.g., blood, plasma, saline) to patients using an IV catheter that is connected though an arrangement of flexible tubing and fittings, commonly referred to as an “IV set,” to a source of fluid, for example, an IV blood bag. During operation, medical fluid may be required quickly at greatly increased flow rates as shorter times to blood transfusions have been associated with decreased death risk in trauma patients. Typical IV sets use a cylindrical hand pump that is squeezed by hand to rapidly increase fluid flow rate, resulting in muscle fatigue.

For these reasons, it is desirable to provide an IV set hand pump with an internal corkscrew member to reduce hand muscle fatigue and to increase the volume of fluid pushed out by the hand pump.

### SUMMARY

In one or more embodiments, an IV set corkscrew hand pump comprises: a body; a corkscrew member disposed in the body; an inlet port disposed on a first end of the body; an outlet port disposed on a second end of the body; and a drive mechanism coupled to the corkscrew member, the drive mechanism configured to cause the corkscrew mechanism to turn within the body.

In one or more embodiments, an IV set comprises: an infusion component; an IV tube coupled to the infusion component; and an IV set corkscrew hand pump coupled to the IV tube, the IV set corkscrew hand pump comprising: a body; a corkscrew member disposed in the body; an inlet port disposed on a first end of the body; an outlet port disposed on a second end of the body; and a drive mechanism coupled to the corkscrew member, the drive mechanism configured to cause the corkscrew mechanism to turn within the body.

In one or more embodiments, a method of operating an IV set corkscrew hand pump with an IV set comprises: coupling an inlet port of the IV set corkscrew hand pump to a fluid source via a first IV tube; coupling an outlet port of the IV set corkscrew hand pump to a fluid delivery device via a second IV tube; starting a flow of fluid from the fluid source into the IV set corkscrew hand pump; and rotating a corkscrew member disposed in a body of the IV set corkscrew hand pump at a determined speed via a drive mechanism coupled to the corkscrew member to pump the fluid from the fluid source through the IV set to the fluid delivery device at a fluid flow rate that exceeds a maximum gravity fluid flow rate of the IV set, wherein the drive mechanism is one of a hand crank assembly and a motor coupling.

The foregoing and other features, aspects and advantages of the disclosed embodiments will become more apparent from the following detailed description and accompanying drawings.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a perspective view of an example patient care system having four fluid infusion pumps, each of which is connected to a respective fluid supply for pumping the contents of the fluid supply to a patient.

FIG. 2 depicts a top view of a typical assembled IV infusion set with a hand pump.

FIG. 3 depicts a front view of an IV set corkscrew hand pump with hand crank, according to aspects of the disclosure.

FIG. 4 depicts a front view of an IV set corkscrew hand pump with motor connector, according to aspects of the disclosure.

FIG. 5 depicts a perspective view of an IV set with the IV set corkscrew hand pump of FIG. 3, according to aspects of the disclosure.

FIG. 6 depicts a perspective view of the IV set corkscrew hand pump of FIG. 3 connecting an infusion pump to a patient, according to aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

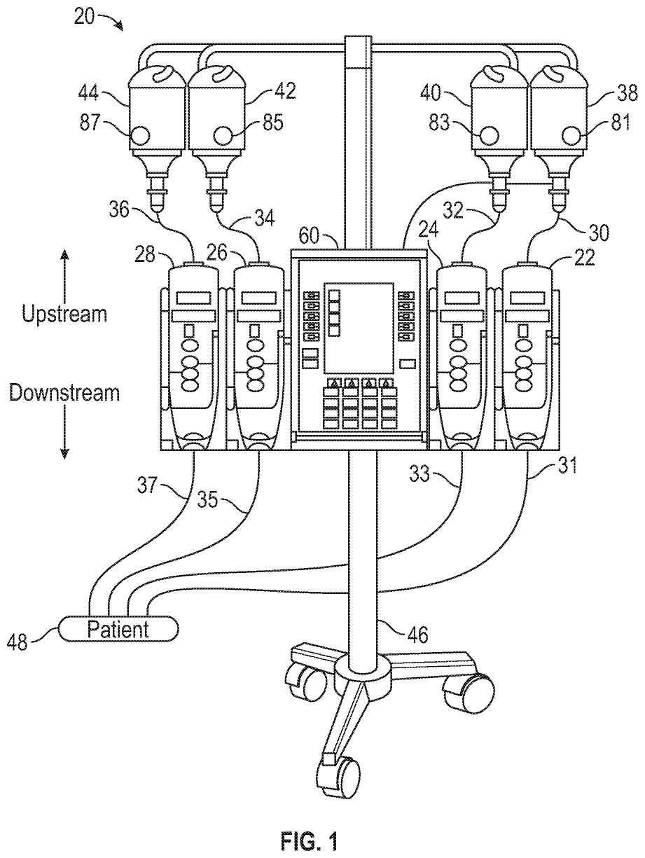

Referring now in more detail to the drawings in which like reference numerals refer to like or corresponding elements among the several views, there is shown in FIG. 1 a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a pump control unit 60.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typical infusion sets may also be gravity sets that do not require use of an infusion pump. For example, any of fluid supplies 38, 40, 42, and 44 may be directly connected to the patient 48 via a gravity IV set, wherein gravity causes the fluid to flow through the infusion set and into the patient 48 without the aid of a pump.

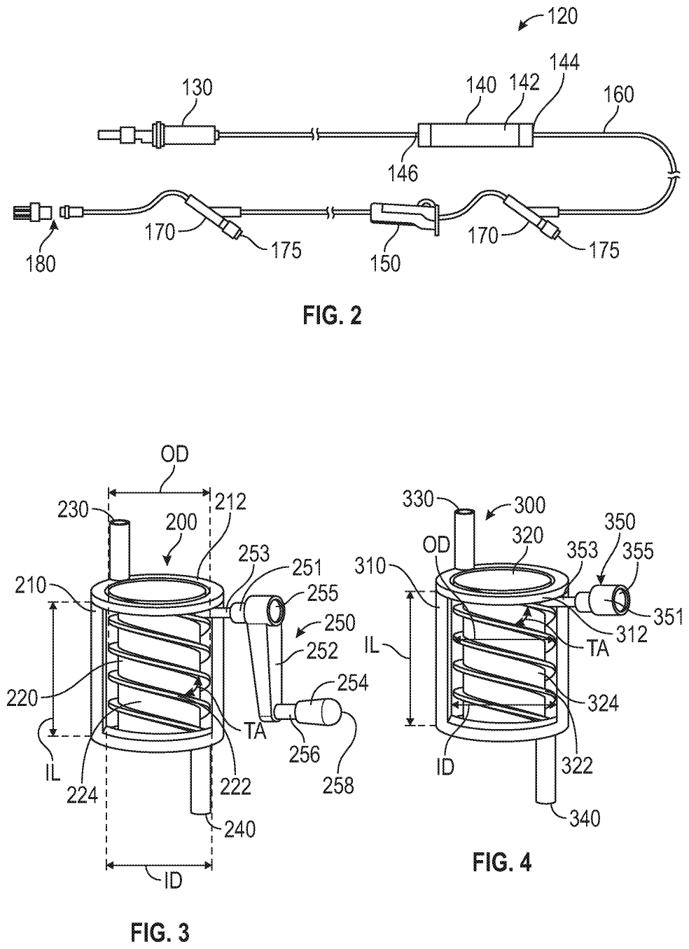

Typically, medical fluid administration sets have more parts than are shown in FIG. 1, such as those shown in FIG. 2. IV sets may be formed from any combination of infusion components and tubing. Typically, the infusion components and tubing are disposable products that are used once and then discarded. The infusion components and tubing may be formed from any suitable material (e.g., plastic, silicone, rubber), many or all of which are clear or translucent so that the fluid flow or levels inside can be seen.

As shown in FIG. 2, an IV set 120 may include a drip chamber 130, a hand pump 140 and a roller clamp 150 connected together by tubing 160. The IV set 120 may also include a Y-site 170 having a Y-shaped junction with a needleless connector 175, as well as a luer lock connector 180 at the end of the IV set 120. The luer lock connector 180 may be used for connection to a catheter inserted into a patient, for example. The IV set 120 may include additional infusion components and may be formed of any combination of components and the tubing 160.

In use, IV set 120 is connected to an IV fluid bag (e.g., blood bag) via the drip chamber 130 and the luer lock connector 180 is connected to a catheter that is placed into a vein of a patient. Thus, fluid flows from the fluid bag through the drip chamber 130 to the hand pump 140 and through the remainder of the IV set 120 and out of the luer lock connector 180. As the hand pump 140 is squeezed, the volume of fluid contained within a body 142 of the hand pump 140 is forced out an outlet port 144 and downstream through the IV set 120. When the hand pump 140 is released (e.g., stop squeezing), the body 142 of the hand pump 140 reinflates and draws in a new volume of fluid through an inlet port 146. This squeezing cycle can be repeated as often as necessary to move fluid from one or more fluid bags into the patient as quickly as possible.

The hand pump 140 is a typical flexible cylinder that is squeezed by a user continuously with one hand and released before activating or squeezing it again. However, the smooth cylindrical shape of the hand pump 140 provides a non-ergonomic shape to be gripped by a hand and no internal features to promote downstream fluid flow. Also, the squeezable cylinder shape geometry is often uncomfortable for prolonged use and may quickly lead to user hand fatigue. In addition, depending on the material the hand pump 140 is made of, it may take a long time to recoil and/or require extra squeeze strength to manipulate.

In aspects of the disclosure, a cylindrical hand pump may be replaced by a corkscrew hand pump having a body with an internal corkscrew member coupled to either a hand crank or a motor. The internal corkscrew member provides for pulling fluid within the hand pump in a downstream direction when rotated by the hand crank or the motor. This decreases user hand fatigue from pumping fluid and speeds up the refill cycle in which new fluid is pulled into the body of the hand pump. Also, unlike the cylindrical hand pump 140, the corkscrew hand pump provides an increased fluid flow rate by rotating the internal corkscrew member faster instead of having to manually squeeze the hand pump more rapidly by hand, as well as providing a more continuous fluid flow as opposed to bursts of fluid flow.

As shown in FIG. 3, an IV set corkscrew hand pump 200 is provided according to aspects of the disclosure. IV set corkscrew hand pump 200 may be in the form of a hand crank design including a body 210, a corkscrew member 220, an inlet port 230, an outlet port 240 and a hand crank 250 (e.g., drive mechanism).

The body 210 may be formed of any suitable material (e.g., polymer, rubber, plastic) that is configured to be in contact with a medical fluid (e.g., saline, blood, plasma). The material of the body 210 may be flexible or inflexible, and may be any single material or combination of materials selected or designed to provide a desired amount of flexibility/inflexibility and life-cycle usage.

In aspects of the disclosure, the cylindrical shape of the body 210 provides curved interior surfaces having an inner diameter ID configured to match an outer diameter OD of the corkscrew member 220, which may decrease or eliminate upstream backflow of fluid within the body 210 and/or dead areas that retain fluid during fluid outflow from the body 210. For example, the cylindrical shape of the corkscrew member 220 may closely fit within the cylinder shape of the body 210 to provide a complete path for the fluid to only flow down within the body 210. By contrast, when the cylindrical hand pump 140 is squeezed, some of the fluid may be forced back upstream within the body. Thus, the IV set corkscrew hand pump 200 may provide for a higher volume of fluid to be forced out of the body 210 with each cycle.

In aspects of the disclosure, the corkscrew member 220 may be disposed within the body 210 along a portion of or the entirety of the interior axial length IL of the body 210. For example, if the corkscrew member 220 extends throughout the entire interior axial length IL of the body 210, then there may be no dead spaces within the body 210 in which fluid may remain static or escape the downstream urging of the rotating corkscrew member 220. The corkscrew member 220 may have threads 222 disposed on a core 224.

In aspects of the disclosure, the diameter of the threads 222 may be sized to have an outer diameter OD that is slightly less than the inner diameter ID of the body 210 in order to maximize the fit between the corkscrew member 220 and the body 210 while providing for the corkscrew member 220 to rotate within the body 210 without binding. In aspects of the disclosure, the threads 222 may be provided with any of a range of smaller outer diameters OD than the inner diameter ID of the body 210, which may change the flow channel and/or flow rate of the fluid within the body 210 and/or the flow rate of the fluid exiting the outlet port 240 per revolution of the corkscrew member 220. For example, the outer diameters OD of the threads 222 may match the tubing diameter so as not to be a flow restrictor when not cranking (e.g., under just gravity flow). In aspects of the disclosure, the distance or spacing between the threads 222 (e.g., thread density) may be selected to provide a desired flow channel and/or flow rate within the body 210.

In aspects of the disclosure, the thread angle TA with respect to the core 224 may be selected to provide a desired flow channel or flow rate of fluid within the body 210. For example, as shown in FIG. 3 the thread angle TA is close to or equal to 90 degrees (e.g., orthogonal). However, the thread angle TA may be any suitable angle (e.g., 45 degrees, 60 degrees).

In aspects of the disclosure, the hand crank 250 may extend radially outward from an outer surface 212 of the body 210 and be coupled to the corkscrew member 220. The hand crank 250 may include a driveshaft 251 having a first end 253 coupled to the corkscrew member 220 and a second end 255 coupled to a crankshaft 252 and a grip handle 254 sized and shaped to be gripped by a user's hand or fingers. The grip handle 254 may include a shaft 256 and a sleeve 258 disposed on a portion of the shaft 256. The sleeve 258 may be a soft and/or padded material configured to be ergonomically comfortable to grip. For example, the sleeve 258 may be a soft material (e.g., rubber, foam) overmolded onto the shaft 256 or formed separately and slid onto the shaft 256.

In aspects of the disclosure, the length of the crankshaft 252 may be configured to provide a suitable flow rate of the fluid within the body 210 and/or a flow rate of the fluid exiting the outlet port 240 per revolution of the hand crank 250. In aspects of the disclosure, diameters or gearing of the coupling (e.g., driveshaft 251) between the corkscrew member 220 and the hand crank 250 may be sized and shaped to provide a suitable flow rate of the fluid within the body 210 and/or a flow rate of the fluid exiting the outlet port 240 per revolution of the hand crank 250. In aspects of the disclosure, any or all of the length of the corkscrew member 220, the outer diameter OD of the threads 222, the thread angle TA of the threads 222, the spacing or density of the threads 222, the configuration of the coupling between the corkscrew member 220 and the hand crank 250 and the length of the crankshaft 252 may be configured or varied independently or in combination in order to obtain the desired fluid flow rate through and/or from the IV set corkscrew hand pump 200 (e.g., exiting downstream of the IV set corkscrew hand pump 200). In aspects of the disclosure, either or both the inlet port 230 and outlet port 240 may be configured as a luer type connector such that the IV set corkscrew hand pump 200 may be quickly added to an IV set if needed.

As shown in FIG. 4, an IV set corkscrew hand pump 300 is provided according to aspects of the disclosure. IV set corkscrew hand pump 300 may be in the form of a motorized design including a body 310, a corkscrew member 320, an inlet port 330, an outlet port 340 and a motor coupling 350.

The body 310 may be formed of any suitable material (e.g., polymer, rubber, plastic) that is configured to be in contact with a medical fluid (e.g., saline, blood, plasma). The material of the body 310 may be flexible or inflexible, and may be any single material or combination of materials selected or designed to provide a desired amount of flexibility/inflexibility and life-cycle usage.

In aspects of the disclosure, the cylindrical shape of the body 310 provides curved interior surfaces having an inner diameter ID configured to match an outer diameter OD of the corkscrew member 320, which may decrease or eliminate upstream backflow of fluid within the body 310 and/or dead areas that retain fluid during fluid outflow from the body 310. For example, the cylindrical shape of the corkscrew member 320 may closely fit within the cylinder shape of the body 310 to provide a complete path for the fluid to only flow down within the body 310. By contrast, when the cylindrical hand pump 140 is squeezed, some of the fluid may be forced back upstream within the body. Thus, the IV set corkscrew hand pump 300 may provide for a higher volume of fluid to be forced out of the body 310 with each cycle.

In aspects of the disclosure, the corkscrew member 320 may be disposed within the body 310 along a portion of or the entirety of the interior axial length IL of the body 310. For example, if the corkscrew member 320 extends throughout the entire interior axial length IL of the body 310, then there may be no dead spaces within the body 310 in which fluid may remain static or escape the downstream urging of the rotating corkscrew member 320. The corkscrew member 320 may have threads 322 disposed on a core 324.

In aspects of the disclosure, the diameter of the threads 322 may be sized to have an outer diameter OD that is slightly less than the inner diameter ID of the body 310 in order to maximize the fit between the corkscrew member 320 and the body 310 while providing for the corkscrew member 320 to rotate within the body 310 without binding. In aspects of the disclosure, the threads 322 may be provided with any of a range of smaller outer diameters OD than the inner diameter ID of the body 310, which may change the flow channel and/or flow rate of the fluid within the body 310 and/or the flow rate of the fluid exiting the outlet port 340 per revolution of the corkscrew member 320. In aspects of the disclosure, the distance or spacing between the threads 322 (e.g., thread density) may be selected to provide a desired flow channel and/or flow rate within the body 310.

In aspects of the disclosure, the thread angle TA with respect to the core 324 may be selected to provide a desired flow channel or flow rate of fluid within the body 310. For example, as shown in FIG. 4 the thread angle TA is close to or equal to 90 degrees (e.g., orthogonal). However, the thread angle TA may be any suitable angle (e.g., 45 degrees, 60 degrees).

In aspects of the disclosure, the motor coupling 350 may extend radially outward from an outer surface 312 of the body 310 and be coupled to the corkscrew member 320. The motor coupling 350 may be sized and shaped to be suitably coupled with a desired motor (e.g. motor shaft). The motor coupling 350 may include a driveshaft 351 having a first end 353 coupled to the corkscrew member 220 and a second end 355 configured to be coupled to a motor.

In aspects of the disclosure, the speed of rotation of the motor coupling 350 (e.g., motor speed) may be configured to provide a suitable flow rate of the fluid within the body 310 and/or a flow rate of the fluid exiting the outlet port 340. In aspects of the disclosure, diameters or gearing of the coupling between the corkscrew member 320 and the motor coupling 350 may be sized and shaped to provide a suitable flow rate of the fluid within the body 310 and/or a flow rate of the fluid exiting the outlet port 340 per revolution of the motor coupling 350. In aspects of the disclosure, any or all of the length of the corkscrew member 320, the outer diameter OD of the threads 322, the thread angle TA of the threads 322, the spacing or density of the threads 322, the configuration of the coupling between the corkscrew member 320 and the motor coupling 350 and the speed of rotation of the motor coupling 350 may be configured or varied independently or in combination in order to obtain the desired fluid flow rate through and/or from the IV set corkscrew hand pump 300.

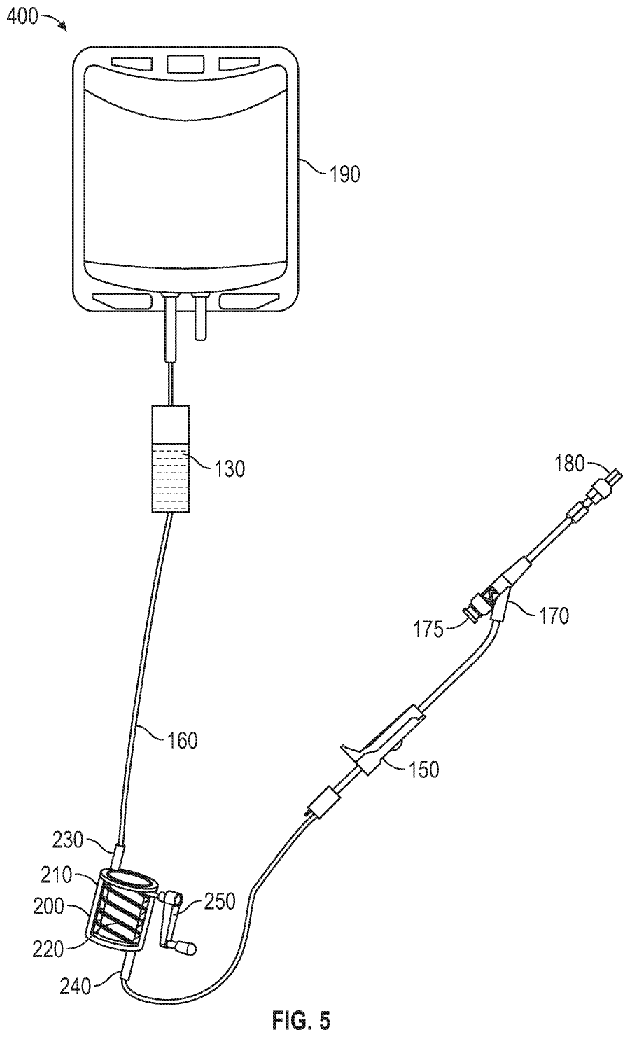

As shown in FIG. 5, the IV set corkscrew hand pump 200 may be part of an IV set 400. The IV set 400 may include a fluid bag 190, a drip chamber 130, an IV set hand pump 200, a roller clamp 150, a Y-junction 170 having a needleless port 175 and a luer connector 180, all coupled together by IV tubing 160. IV set corkscrew hand pump 300 or any other desired IV set corkscrew hand pump may be used in place of IV set corkscrew hand pump 200 in the IV set 400, for example.

In aspects of the disclosure, the IV set corkscrew hand pump 200, 300 may be a passive in-line device when not activated (e.g., being squeezed) in which fluid from the fluid bag 190 is received via the drip chamber 130 into the inlet port 230, 330 and the fluid passes through the body 210, 310 and out the outlet port 240, 340 at a flow rate controlled by any of gravity, the volume of fluid in the fluid bag 190 and/or the roller clamp 150. In use, when the corkscrew member 220, 320 is rotated via the hand crank 250 or the motor coupling 350, the volume of fluid that has accumulated within the body 210, 310 during the passive flow phase may be fully forced out of the outlet port 240, 340 very rapidly over one or more revolutions of the corkscrew member 220, 320.

This rotation of the corkscrew member 220, 320 may cause a suction effect upstream, thus causing more fluid to flow from the fluid bag 190/drip chamber 130 into the IV set corkscrew hand pump 200, 300 quickly (e.g., more quickly than gravity allows). Thus, continuous rotation of the corkscrew member 220, 320 may cause all of the fluid to be rapidly pulled out of the fluid bag 190 and pushed into a desired receptacle (e.g., a patient's arm).

In aspects of the disclosure, the IV set corkscrew hand pump 200, 300 may be a disposable component of the IV set 400. For example, the IV set corkscrew hand pump 200, 300 may be an integral component of the IV set 400 where the body 210, 310 may be coupled in line via IV tubing 160 to the drip chamber 130 and the roller clamp 150. Accordingly, the disposable IV set corkscrew hand pump 200, 300 would be used for the life of the IV set only (e.g., 24 hours, 72 hours, 7 days), whereafter the IV set corkscrew hand pump 200, 300 would be disposed of along with the associated IV set 400. In aspects of the disclosure, the IV set corkscrew hand pump 200, 300 may be coupled anywhere in line within the IV set 400, such as directly to the fluid bag 190 in place of a drip chamber 130 or close to the luer connector 180, for example.

In use, the IV set 400 with the disposable IV set corkscrew hand pump 200, 300 is coupled to a fluid container (e.g., fluid bag 190) containing a medical fluid (e.g., blood). In an unactuated state, IV set corkscrew hand pump 200, 300 has a static corkscrew member 220, 320, thus allowing the fluid to flow through the IV set 400 at a rate set by a flow controller (e.g., roller clamp 150). When quicker fluid flow is needed, the corkscrew member 220, 320 may be rotated by hand via the hand crank 250 or mechanically via a motor coupled to the motor coupling 350, thus squeezing the fluid out of the body 210, 310 quickly.

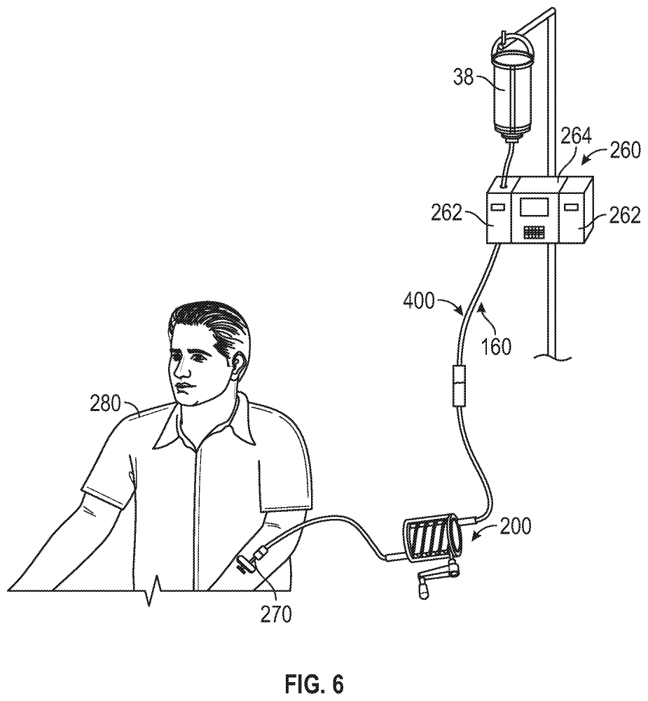

As shown in FIG. 6, the IV set 400 and IV set corkscrew hand pump 200, 300 may be coupled to a fluid source 38, to an infusion pump 260 system having two infusion pumps 262 and a controller 264, and to a catheter 270 inserted into a patient 280. Here, if the maximum fluid flow rate from the infusion pump system 260 is not sufficient, the IV set corkscrew hand pump 200, 300 may be activated (e.g., hand cranked, motor turning) to force the fluid to flow more quickly. For example, the IV set 400 may be quickly uncoupled from the infusion pump system 260 before using the IV set corkscrew hand pump 200, 300 and/or the infusion pump system 260 may be set to neutral to allow unimpeded fluid flow from the fluid source 38 before using the IV set corkscrew hand pump 200, 300.

In aspects of the disclosure, the IV set corkscrew hand pump 200, 300 may include any suitable fastener to couple the IV set corkscrew hand pump 200, 300 to an IV pole 46, a bed, an operating table and the like. For example, the fastener may be a cradle, a hangar, a hook, Velcro ®, adhesive, and/or any other suitable fastener. In aspects of the disclosure, the IV set corkscrew hand pump 200, 300 may be configured to simply hang in line with an IV set (e.g., IV set 400) via IV tubing (e.g., IV tubing 160). Here, the IV set corkscrew hand pump 200, 300 may be positioned on the IV set to maximize accessibility and/or to keep the IV set corkscrew hand pump 200, 300 out of specific work areas (e.g., right above patient).

In aspects of the disclosure, the geometry and/or shape of the body 210, 310 may be varied based on hand size and/or desired flow volume. For example, there may be small, medium and large sizes for use by different sized users. In aspects of the disclosure, the flow rates may be configured based on size/shape of the body 210, 310, diameter of the inlet port 230, 330 and outlet port 240, 340, and/or flow channels defined by the threads 222, 322 and the core 224, 324.

In aspects of the disclosure, drugs may be administered to a patient quickly via a corkscrew hand pump that forces the drugs via the IV line at the rate with which the corkscrew member 220, 320 is rotating.

In some embodiments according to the disclosure, an IV set corkscrew hand pump comprises: a body; a corkscrew member disposed in the body; an inlet port disposed on a first end of the body; an outlet port disposed on a second end of the body; and a drive mechanism coupled to the corkscrew member, the drive mechanism configured to cause the corkscrew mechanism to turn within the body.

In aspects of the disclosure, the drive mechanism is a hand crank assembly extending outward from an exterior surface of the body. In aspects of the disclosure, the hand crank assembly comprises: a driveshaft having a first end coupled to the corkscrew member; a crankshaft coupled to a second end of the driveshaft; and a grip handle coupled to the crankshaft. In aspects of the disclosure, the drive mechanism is a motor coupling extending outward from an exterior surface of the body. In aspects of the disclosure, the motor coupling comprises: a driveshaft having a first end coupled to the corkscrew member; and a second end configured to be coupled to a motor. In aspects of the disclosure, the body comprises a cylindrical shape having an inner diameter, wherein the corkscrew member comprises a cylindrical shape having an outer diameter sized to fit just within the inner diameter of the body, and wherein the corkscrew member is configured to turn within the body without binding.

In aspects of the disclosure, the corkscrew member extends along an entire interior axial length of the body. In aspects of the disclosure, the corkscrew member comprises: an axial core; and radial threads disposed on the axial core. In aspects of the disclosure, a spacing between the radial threads is configured to provide a determined flow channel within the body. In aspects of the disclosure, a spacing between the radial threads is configured to provide a determined fluid flow rate downstream from the body. In aspects of the disclosure, the radial threads extend from the axial core at a thread angle configured to provide a determined flow channel within the body. In aspects of the disclosure, the radial threads extend from the axial core at a thread angle configured to provide a determined fluid flow rate downstream from the body. In aspects of the disclosure, a length of the axial core is configured to provide a determined fluid flow rate downstream from the body. In aspects of the disclosure, an outer diameter of the radial threads is configured to provide a determined fluid flow rate downstream from the body. In aspects of the disclosure, one of the inlet port and the outlet port comprises a luer connector.

In some embodiments according to the disclosure, an IV set comprises: an infusion component; an IV tube coupled to the infusion component; and an IV set corkscrew hand pump coupled to the IV tube, the IV set corkscrew hand pump comprising: a body; a corkscrew member disposed in the body; an inlet port disposed on a first end of the body; an outlet port disposed on a second end of the body; and a drive mechanism coupled to the corkscrew member, the drive mechanism configured to cause the corkscrew mechanism to turn within the body.

In aspects of the disclosure, the drive mechanism is a hand crank assembly comprising: a driveshaft having a first end coupled to the corkscrew member; a crankshaft coupled to a second end of the driveshaft; and a grip handle coupled to the crankshaft, wherein a first speed of rotation of the driveshaft by hand via the grip handle and the crankshaft is configured to rotate the corkscrew member within the body at a second speed of rotation to cause a first flow rate of fluid exiting the outlet port. In aspects of the disclosure, the drive mechanism is a motor coupling comprising: a driveshaft having a first end coupled to the corkscrew member; and a second end configured to be coupled to a motor, wherein a first speed of rotation of the driveshaft by the motor is configured to rotate the corkscrew member within the body at a second speed of rotation to cause a first flow rate of fluid exiting the outlet port.

In some embodiments according to the disclosure, a method of operating an IV set corkscrew hand pump with an IV set comprises: coupling an inlet port of the IV set corkscrew hand pump to a fluid source via a first IV tube; coupling an outlet port of the IV set corkscrew hand pump to a fluid delivery device via a second IV tube; starting a flow of fluid from the fluid source into the IV set corkscrew hand pump; and rotating a corkscrew member disposed in a body of the IV set corkscrew hand pump at a determined speed via a drive mechanism coupled to the corkscrew member to pump the fluid from the fluid source through the IV set to the fluid delivery device at a fluid flow rate that exceeds a maximum gravity fluid flow rate of the IV set, wherein the drive mechanism is one of a hand crank assembly and a motor coupling.

In aspects of the disclosure, the IV set corkscrew hand pump is configured to replace a hand pump bulb, thereby reducing user hand fatigue and increasing fluid flow rate to reduce fluid transfusion time during use of the IV set.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An intravenous (IV) set corkscrew hand pump comprising:
a body;
a corkscrew member disposed in the body;
an inlet port disposed on a first end of the body;
an outlet port disposed on a second end of the body; and
a drive mechanism coupled to the corkscrew member, the drive mechanism configured to cause the corkscrew mechanism to turn within the body.

2. The IV set corkscrew hand pump of claim 1, wherein the drive mechanism is a hand crank assembly extending outward from an exterior surface of the body.

3. The IV set corkscrew hand pump of claim 2, wherein the hand crank assembly comprises:
a driveshaft having a first end coupled to the corkscrew member;
a crankshaft coupled to a second end of the driveshaft; and
a grip handle coupled to the crankshaft.

4. The IV set corkscrew hand pump of claim 1, wherein the drive mechanism is a motor coupling extending outward from an exterior surface of the body.

5. The IV set corkscrew hand pump of claim 4, wherein the motor coupling comprises:
a driveshaft having a first end coupled to the corkscrew member; and
a second end configured to be coupled to a motor.

6. The IV set corkscrew hand pump of claim 1, wherein the body comprises a cylindrical shape having an inner diameter, wherein the corkscrew member comprises a cylindrical shape having an outer diameter sized to fit just within the inner diameter of the body, and wherein the corkscrew member is configured to turn within the body without binding.

7. The IV set corkscrew hand pump of claim 1, wherein the corkscrew member extends along an entire interior axial length of the body.

8. The IV set corkscrew hand pump of claim 1, wherein the corkscrew member comprises:
an axial core; and
radial threads disposed on the axial core.

9. The IV set corkscrew hand pump of claim 8, wherein a spacing between the radial threads is configured to provide a determined flow channel within the body.

10. The IV set corkscrew hand pump of claim 8, wherein a spacing between the radial threads is configured to provide a determined fluid flow rate downstream from the body.

11. The IV set corkscrew hand pump of claim 8, wherein the radial threads extend from the axial core at a thread angle configured to provide a determined flow channel within the body.

12. The IV set corkscrew hand pump of claim 8, wherein the radial threads extend from the axial core at a thread angle configured to provide a determined fluid flow rate downstream from the body.

13. The IV set corkscrew hand pump of claim 8, wherein a length of the axial core is configured to provide a determined fluid flow rate downstream from the body.

14. The IV set corkscrew hand pump of claim 8, wherein an outer diameter of the radial threads is configured to provide a determined fluid flow rate downstream from the body.

15. The IV set corkscrew hand pump of claim 1, wherein one of the inlet port and the outlet port comprises a luer connector.

16. An intravenous (IV) set comprising:
an infusion component;
an IV tube coupled to the infusion component; and
an IV set corkscrew hand pump coupled to the IV tube, the IV set corkscrew hand pump comprising:
a body;
a corkscrew member disposed in the body;
an inlet port disposed on a first end of the body;
an outlet port disposed on a second end of the body; and
a drive mechanism coupled to the corkscrew member, the drive mechanism configured to cause the corkscrew mechanism to turn within the body.

17. The IV set of claim 16, wherein the drive mechanism is a hand crank assembly comprising:
a driveshaft having a first end coupled to the corkscrew member;
a crankshaft coupled to a second end of the driveshaft; and
a grip handle coupled to the crankshaft,
wherein a first speed of rotation of the driveshaft by hand via the grip handle and the crankshaft is configured to rotate the corkscrew member within the body at a second speed of rotation to cause a first flow rate of fluid exiting the outlet port.

18. The IV set of claim 16, wherein the drive mechanism is a motor coupling comprising:
a driveshaft having a first end coupled to the corkscrew member; and
a second end configured to be coupled to a motor,
wherein a first speed of rotation of the driveshaft by the motor is configured to rotate the corkscrew member within the body at a second speed of rotation to cause a first flow rate of fluid exiting the outlet port.

19. A method of operating an intravenous (IV) set corkscrew hand pump with an IV set, the method comprising:
coupling an inlet port of the IV set corkscrew hand pump to a fluid source via a first IV tube;
coupling an outlet port of the IV set corkscrew hand pump to a fluid delivery device via a second IV tube;
starting a flow of fluid from the fluid source into the IV set corkscrew hand pump; and
rotating a corkscrew member disposed in a body of the IV set corkscrew hand pump at a determined speed via a drive mechanism coupled to the corkscrew member to pump the fluid from the fluid source through the IV set to the fluid delivery device at a fluid flow rate that exceeds a maximum gravity fluid flow rate of the IV set,
wherein the drive mechanism is one of a hand crank assembly and a motor coupling.

20. The method of claim 19, wherein the IV set corkscrew hand pump is configured to replace a hand pump bulb, thereby reducing user hand fatigue and increasing fluid flow rate to reduce fluid transfusion time during use of the IV set.
